(19) United States
(12) Patent Application Publication
(10) Pub. No.: US 2022/0248781 A1
(43) Pub. Date: Aug. 11, 2022

(54) Sanitary Safety Mask Assembly

(71) Applicant: Laura Mann, Hull, MA (US)

(72) Inventor: Laura Mann, Hull, MA (US)

(21) Appl. No.: 17/173,994

(22) Filed: Feb. 11, 2021

(51) Int. Cl. A41D 13/11; A61L 9/04; A61L 9/12

(52) U.S. Cl. CPC A41D 13/1192; A41D 13/1161; A41D 13/113; A61L 2209/15; A61L 9/12; A41D 2400/36; A61L 9/042

(57) ABSTRACT

A sanitary safety mask assembly for inhibiting the airborne transmission of infectious disease includes a mask that is wearable over a user's face. The mask is comprised of a microbe blocking material to inhibit airborne transmission of infectious diseases. A pair of straps is each coupled to the mask and each of the straps forms a closed loop for positioning around a respective one of the user's ears. An air valve is integrated into the mask, and the air valve is actuatable into a closed condition or an open condition. A pad is provided that is infused with a chemical aroma such that the pad releases the chemical aroma as a vapor. The pad is insertable into the air valve to release the vapor of the chemical aroma into the air that is breathed by the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to safety mask devices and more particularly pertains to a new safety mask device for inhibiting the airborne transmission of infectious disease. The safety mask includes an air valve and a pad that is soaked with a chemical aroma which can be placed in the air valve. In this way a user of the mask can inhale the chemical aroma while the user is wearing the mask.

### (2) Description of Related Art Including Information Disclosed Under 37 CFR 1.97 and 1.98

The prior art relates to safety mask devices including a variety of mask devices that have an aromatic element integrated therein. The prior art also discloses a variety of aromatherapy dispensers for releasing a pleasing aroma. In no instance does the prior art disclose a mask with an air valve and a pad which is soaked with a chemical aroma that can be inserted into the air valve.

### BRIEF SUMMARY OF THE INVENTION

An embodiment of the disclosure meets the needs presented above by generally comprising a mask that is wearable over a user's face. The mask is comprised of a microbe blocking material to inhibit airborne transmission of infectious diseases. A pair of straps is each coupled to the mask and each of the straps forms a closed loop for positioning around a respective one of the user's ears. An air valve is integrated into the mask, and the air valve is actuatable into a closed condition or an open condition. A pad is provided that is infused with a chemical aroma such that the pad releases the chemical aroma as a vapor. The pad is insertable into the air valve to release the vapor of the chemical aroma into the air that is breathed by the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### ) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a sanitary safety mask assembly according to an embodiment of the disclosure.

FIG. 2 is a perspective view of an air valve and a pad of an embodiment of the disclosure.

FIG. 3 is a back cut-away view of an embodiment of the disclosure.

FIG. 4 is a cross sectional view taken along line 4-4 of FIG. 1 of an embodiment of the disclosure.

FIG. 5 is a perspective in-use view of an embodiment of the disclosure.

### DETAILED DESCRIPTION OF THE INVENTION

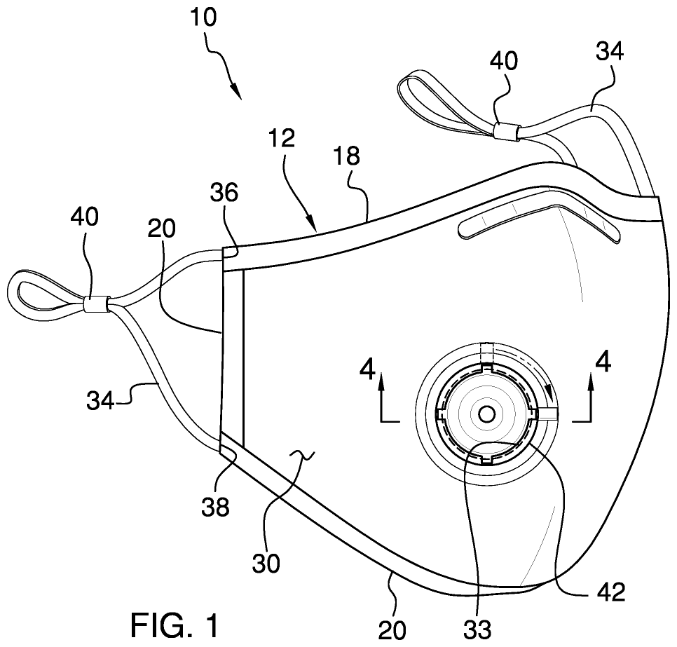

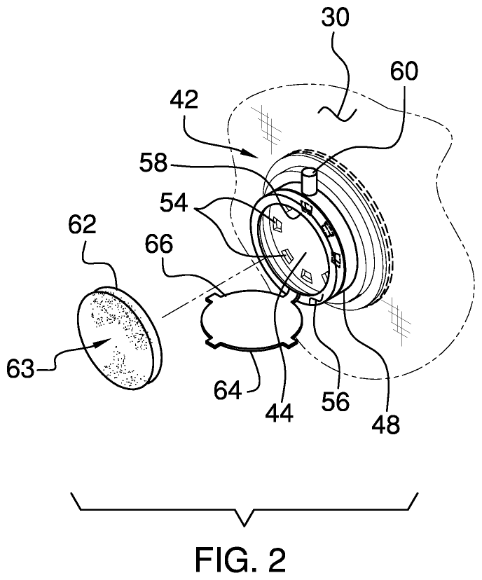

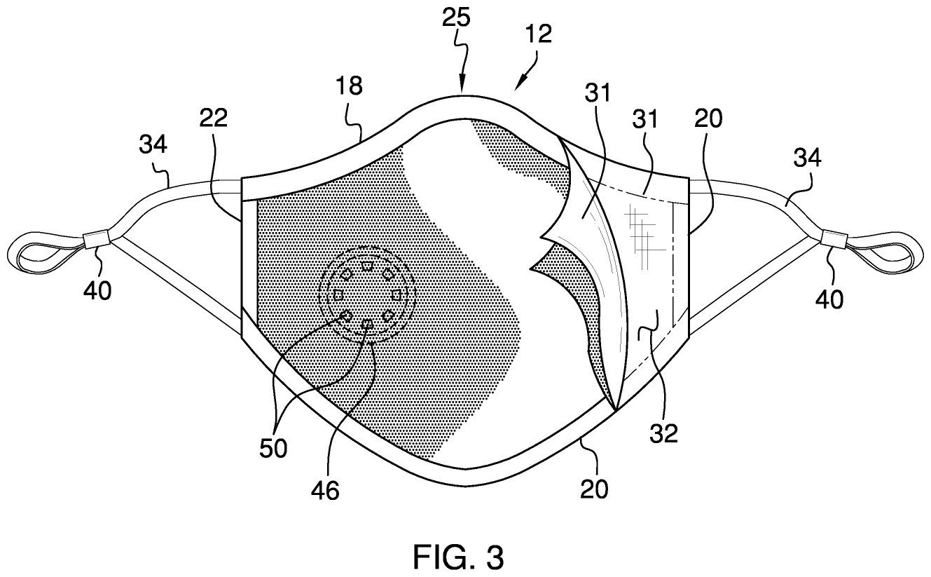

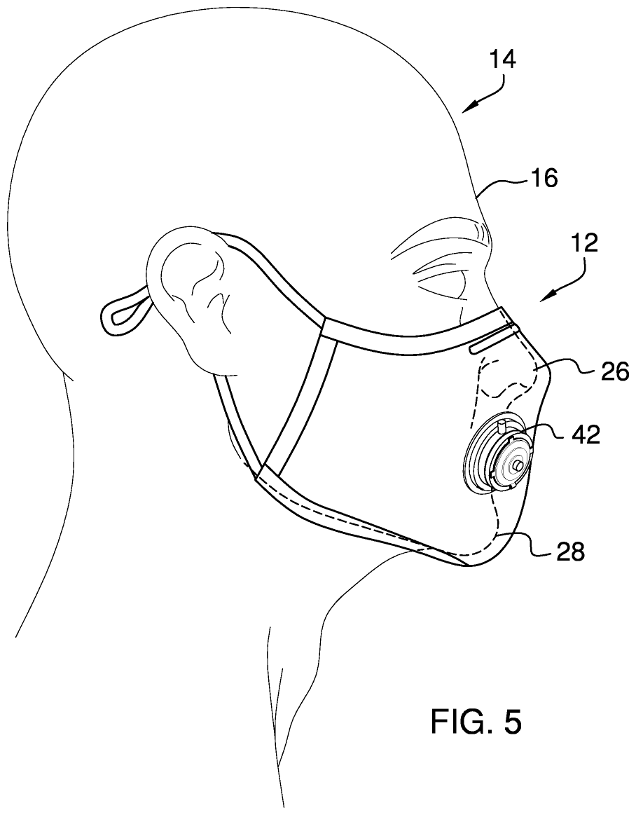

With reference now to the drawings, and in particular to FIGS. 1 through 5 thereof, a new safety mask device embodying the principles and concepts of an embodiment of the disclosure and generally designated by the reference numeral 10 will be described.

As best illustrated in FIGS. 1 through 5, the sanitary safety mask assembly 10 generally comprises a mask 12 that is wearable by a user 14 to cover the user's face 16. The mask 12 is comprised of a microbe blocking material to inhibit airborne transmission of infectious diseases. Additionally, the mask 12 is comprised of an air permeable material to facilitate the user 14 to breathe when the user 14 wears the mask 12. The microbe blocking material and the air permeable material may comprise a polypropylene material, a flannel material or other similar material that is approved for use for protection of infectious diseases. The mask 12 may also have silver woven into the fabric of which the mask 12 is comprised for enhancing the anti-bacterial properties of the mask 12. The mask 12 may additionally be comprised of a graphene material for inhibiting microbes from passing through the mask 12.

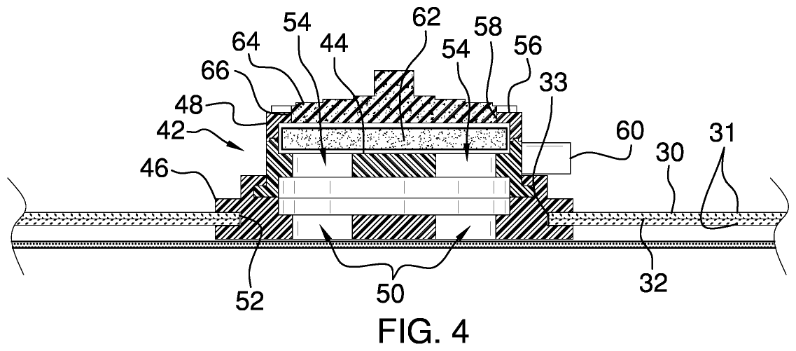

The mask 12 has a top edge 18, a bottom edge 20, a first lateral edge 22 and a second lateral edge 24. The top edge 18 slants upwardly toward a peak 25 between the first lateral edge 22 and the second lateral edge 24 to accommodate the user's nose 26 when the mask 12 is worn. The bottom edge 20 curves downwardly between the first lateral edge 22 and the second lateral edge 24 to accommodate the user's chin 28 when the mask 12 is worn. The mask 12 has a front surface 30 and a back surface 32, and the mask 12 has an air passage 33 extending through the front surface 30 and the back surface 32 to pass air therethrough. As is most clearly shown in FIG. 4, the mask 12 may comprise a pair of layers 31 that is bonded together, and each of the layers 31 may define a respective front surface 30 and back surface 32 of the mask 12.

A pair of straps 34 is provided and each of the straps 34 is coupled to the mask 12. Each of the straps 34 forms a closed loop with the mask 12 thereby facilitating the straps 34 to be positioned around a respective one of the user 14's ears. Each of the straps 34 has a first end 36 and a second end 38, and the first end 36 and the second end 38 of each of straps 34 is coupled to a respective one of the first lateral edge 22 and the second lateral edge 24 of the mask 12. Thus, each of the straps 34 is positioned on the respective first lateral edge 22 or the second lateral edge 24. Each of the straps 34 includes an adjuster 40 that is slidably positioned thereon for adjusting the size of the closed loop defined by each of the straps 34. In this way the straps 34 can accommodate a variety of sizes of ears. The adjuster 40 may comprise a tube through which the respective strap extends and which is slidable toward or away from the mask 12.

An air valve 42 is integrated into the mask 12 and the air valve 42 has a chamber 44 that is integrated therein. The air valve 42 is actuatable into a closed condition to inhibit air from passing therethrough. Conversely, the air valve 42 is positionable in an open condition to pass air therethrough. The air valve 42 comprises a first portion 46 and a second portion 48 that rotatably engages the first portion 46. The first portion 46 has a plurality of first channels 50 each extending therethrough to pass air therethrough. Additionally, the first portion 46 is positioned in the air passage 33 in the mask 12 having the first portion 46 engaging a bounding edge 52 of the air passage 33.

The second portion 48 has a plurality of second channels 54 each extending therethrough to pass air therethrough. The second portion 48 is rotatable into an open position having each of the second channels 54 being aligned with a respective one of the first channels 50. In this way the air valve 42 can pass air therethrough. The second portion 48 is rotatable into a closed position has each of the second channels 54 being offset with the first channels 50. In this way the air valve 42 inhibits air from passing therethrough. The second portion 48 is rotatable into a plurality of positions between the open position and the closed position. In this way the air valve 42 can pass a minimum amount of air or a maximum amount of air therethrough.

The chamber 44 is integrated into the second portion 48 and the chamber 44 is in fluid communication with each of the second channels 54. The second portion 48 has an exposed surface 56 with respect to the front surface 30 of the mask 12 and the exposed surface 56 has an opening 58 extending into the chamber 44. Additionally, the second portion 48 has a button 60 extending laterally away therefrom which can be gripped by a user 14 for rotating the second portion 48 between the open position and the closed position.

A pad 62 is provided and the pad 62 is infused with a chemical aroma 63 such that the pad 62 releases the chemical aroma 63 as a vapor. The pad 62 may be comprised of wool or other type of absorbent textile and the chemical aroma 63 may be an essential oil of the user's 14 preferred aroma or any other volatile chemical aroma that is non-toxic for human breathing. Additionally, the chemical aroma 63 may comprise an anti-viral compound that can enhance sanitary conditions for the user 14 while the user 14 is wearing the mask 12. The pad 62 is insertable into the chamber 44 in the air valve 42 such that the pad 62 can release the vapor of the chemical aroma 63 into the air is breathed by the user 14.

A cap 64 is removably attachable to the air valve 42 for closing the chamber 44 to retain the pad 62 in the chamber 44. The cap 64 has an outer edge 66 and the cap 64 is positionable in the opening 58 in the exposed surface 56 of the second portion 48 of the air valve 42. The outer edge 66 of the cap 64 frictionally engages a bounding edge 60 of the opening 58 in the exposed surface 56. The cap 64 may be comprised of a resiliently compressible material such as rubber or the like.

In use, the pad 62 is inserted into the chamber 44 and the cap 64 is positioned in the opening 58 in the air valve 42 to close the chamber 44. The button 60 is manipulated to position the second portion 48 of the valve in the desired position for passing the desired amount of air through the air valve 42. In this way the user 14 can inhale the pleasing chemical aroma while the user 14 is wearing the mask 12. The pad 62 is removed and replaced in the chamber 44 when the chemical aroma becomes depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sanitary safety mask assembly having an aromatherapy pouch integrated therein for releasing a therapeutic aroma, said assembly comprising:
a mask being wearable over a user's face, said mask being comprised of a microbe blocking material wherein said mask is configured to inhibit airborne transmission of infectious diseases, said mask being comprised of an air permeable material wherein said mask is configured to facilitate the user to breathe when the user wears said mask;
a pair of straps, each of said straps being coupled to said mask, each of said straps forming a closed loop with said mask wherein each of said straps is configured to be positioned around a respective one of the user's ears;
an air valve being integrated into said mask, said air valve having a chamber being integrated therein, said air valve being actuatable into a closed condition wherein said air valve is configured to inhibit air from passing therethrough, said air valve being positionable in an open condition wherein said air valve is configured to pass air therethrough;
a pad being infused with a chemical aroma such that said pad releases said chemical aroma as a vapor, said pad being insertable into said chamber in said air valve wherein said pad is configured to release said vapor of said chemical aroma into the air being breathed by the user; and
a cap being removably attachable to said air valve for closing said chamber to retain said pad in said chamber.

2. The assembly according to claim 1, wherein said mask has a top edge, a bottom edge, a first lateral edge and a second lateral edge, said top edge slanting upwardly toward a peak between said first lateral edge and said second lateral edge wherein said peak is configured to accommodate the user's nose when said mask is worn, said bottom edge curving downwardly between said first lateral edge and said second lateral edge wherein said bottom edge is configured to accommodate the user's chin when said mask is worn.

3. The assembly according to claim 2, wherein said mask has a front surface and a back surface, said mask having an air passage extending through said front surface and said back surface wherein said air passage is configured to pass air therethrough, said mask comprising a pair of layers being bonded together.

4. The assembly according to claim 2, wherein each of said straps has a first end and a second end, said first end and said second end of each of straps being coupled to a respective one of said first lateral edge and said second lateral edge of said mask such that each of said straps is positioned on said respective first lateral edge or said second lateral edge.

5. The assembly according to claim 4, wherein each of said straps includes an adjuster being slidably positioned thereon for adjusting the size of the closed loop defined by each of said straps wherein said straps are configured to accommodate a variety of sizes of ears.

6. The assembly according to claim 3, wherein said air valve comprises a first portion and a second portion rotatably engaging said first portion, said first portion having a plurality of first channels each extending therethrough wherein said first portion is configured to pass air therethrough, said first portion being positioned in said air passage in said mask having said first portion engaging a bounding edge of said air passage.

7. The assembly according to claim 6, wherein:
said second portion has a plurality of second channels each extending therethrough wherein said second portion is configured to pass air therethrough;
said second portion is rotatable into an open position having each of said second channels being aligned with a respective one of said first channels wherein said air valve is configured to pass air therethrough;
said second portion is rotatable into a closed position having each of said second channels being offset with said first channels wherein said air valve is configured to inhibit air from passing therethrough; and
said second portion is rotatable into a plurality of positions between said open position and said closed position wherein said air valve is configured to pass a minimum amount of air or a maximum amount of air therethrough.

8. The assembly according to claim 7, wherein said chamber is integrated into said second portion, said chamber being in fluid communication with each of said second channels.

9. The assembly according to claim 8, wherein said second portion has an exposed surface with respect to said front surface of said mask, said exposed surface having an opening extending into said chamber.

10. The assembly according to claim 6, wherein said second portion has a button extending laterally away therefrom wherein said button is configured to be gripped by a user for rotating said second portion between said open position and said closed position.

11. A sanitary safety mask assembly having an aromatherapy pouch integrated therein for releasing a therapeutic aroma, said assembly comprising:
a mask being wearable over a user's face, said mask being comprised of a microbe blocking material wherein said mask is configured to inhibit airborne transmission of infectious diseases, said mask being comprised of an air permeable material wherein said mask is configured to facilitate the user to breathe when the user wears said mask, said mask having a top edge, a bottom edge, a first lateral edge and a second lateral edge, said top edge slanting upwardly toward a peak between said first lateral edge and said second lateral edge wherein said peak is configured to accommodate the user's nose when said mask is worn, said bottom edge curving downwardly between said first lateral edge and said second lateral edge wherein said bottom edge is configured to accommodate the user's chin when said mask is worn, said mask having a front surface and a back surface, said mask having an air passage extending through said front surface and said back surface wherein said air passage is configured to pass air therethrough, said mask comprising a pair of layers being bonded together;
a pair of straps, each of said straps being coupled to said mask, each of said straps forming a closed loop with said mask wherein each of said straps is configured to be positioned around a respective one of the user's ears, each of said straps having a first end and a second end, said first end and said second end of each of straps being coupled to a respective one of said first lateral edge and said second lateral edge of said mask such that each of said straps is positioned on said respective first lateral edge or said second lateral edge, each of said straps including an adjuster being slidably positioned thereon for adjusting the size of the closed loop defined by each of said straps wherein said straps are configured to accommodate a variety of sizes of ears;
an air valve being integrated into said mask, said air valve having a chamber being integrated therein, said air valve being actuatable into a closed condition wherein said air valve is configured to inhibit air from passing therethrough, said air valve being positionable in an open condition wherein said air valve is configured to pass air therethrough, said air valve comprising a first portion and a second portion rotatably engaging said first portion, said first portion having a plurality of first channels each extending therethrough wherein said first portion is configured to pass air therethrough, said first portion being positioned in said air passage in said mask having said first portion engaging a bounding edge of said air passage, said second portion having a plurality of second channels each extending therethrough wherein said second portion is configured to pass air therethrough, said second portion being rotatable into an open position having each of said second channels being aligned with a respective one of said first channels wherein said air valve is configured to pass air therethrough, said second portion being rotatable into a closed position having each of said second channels being offset with said first channels wherein said air valve is configured to inhibit air from passing therethrough, said second portion being rotatable into a plurality of positions between said open position and said closed position wherein said air valve is configured to pass a minimum amount of air or a maximum amount of air therethrough, said chamber being integrated into said second portion, said chamber being in fluid communication with each of said second channels, said second portion having an exposed surface with respect to said front surface of said mask, said exposed surface having an opening extending into said chamber, said second portion having a button extending laterally away therefrom wherein said button is configured to be gripped by a user for rotating said second portion between said open position and said closed position;
a pad being infused with a chemical aroma such that said pad releases said chemical aroma as a vapor, said pad being insertable into said chamber in said air valve wherein said pad is configured to release said vapor of said chemical aroma into the air being breathed by the user; and
a cap being removably attachable to said air valve for closing said chamber to retain said pad in said chamber, said cap having an outer edge, said cap being positionable in said opening in said exposed surface of said second portion of said air valve having said outer edge frictionally engaging a bounding edge of said opening in said exposed surface.
